(19) United States
(12) Patent Application Publication
(10) Pub. No.: US 2008/0107715 A1
(43) Pub. Date: May 8, 2008

(54) PHARMACEUTICAL CONTAINING FOOD COMPOSITIONS AND USES THEREOF

(76) Inventors: Eric C. Miller, Los Gatos, CA (US); Scott M. Smith, Redwood Shores, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/556,644

(22) Filed: Nov. 3, 2006

(51) Int. Cl. A61K 47/00

(52) U.S. Cl. 424/439

(57) ABSTRACT

A method of delivering nutrition and one or more pharmaceutical agents to a patient after obesity surgery to enhance the patient's nutritional intake and/or compliance with oral medication(s) may include: providing a food product in an amount suitable for fitting within a stomach of the patient after the obesity surgery, wherein the food product is formulated to include amounts of carbohydrate, fat, protein, vitamins, minerals and electrolytes to satisfy specific nutritional needs of the post-obesity-surgery patient; incorporating into the food product an effective amount of at least one pharmaceutical agent that modulates at least one of appetite, hyperlipidemia, hypertension, diabetes, sleep apnea, degenerative joint disease, or depression to produce a supplemented food product; and administering the supplemented food product to the patient after obesity surgery so as to enhance at least one of the patient's nutritional intake or compliance with oral medication.

### CROSS-REFERENCES TO RELATED APPLICATIONS

This application claims priority to U.S. Provisional Patent Application Ser. No. 60/______ (Original Attorney Docket No. 30288-701.101), entitled “Pharmaceutical Containing Food Compositions and Uses Therefore,” and filed on Nov. 4, 2005, the full disclosure of which is hereby incorporated by reference.

### BACKGROUND OF THE INVENTION

The incidence of obesity and its associated health-related problems have reached epidemic proportions in the United States. See, for example, Mun et al., “Current Status of Medical and Surgical Therapy for Obesity,” Gastroenterology, 120: 669-681 (2001). Recent investigations suggest that the causes of obesity involve a complex interplay of genetic, environmental, psycho-behavioral, endocrine, metabolic, cultural, and socioeconomic factors. Severe obesity is frequently associated with significant comorbid medical conditions, including coronary artery disease, hypertension, type II diabetes mellitus, gallstones, nonalcoholic steatohepatitis, pulmonary hypertension, depression and sleep apnea.

Estimates of the incidence of morbid obesity are approximately 2% of the U.S. population and 0.5% worldwide. Current treatments range from diet, exercise, behavior modification, and pharmacotherapy to various types of surgery, with varying risks and efficacy. In general, nonsurgical modalities, although less invasive, achieve only relatively short-term and limited weight loss in most patients. Surgical treatments for obesity generally include restrictive procedures and combination restrictive/malabsorptive procedures. Restrictive procedures reduce the size of the stomach by banding (such as with the LapBand®), stapling, filling space in the stomach with an inflatable balloon or the like, but attempt to leave the digestive process largely intact. Restrictive/malabsorptive combination procedures reduce both the size of the stomach and the amount of nutrients and calories absorbed by the digestive system. Examples of restrictive/malabsorptive surgical procedures include the Roux-en-Y gastric bypass, duodenal switch and biliopancreatic diversion procedures. For the purposes of this application, restrictive procedures, restrictive/malabsorptive procedures, and all other surgical procedures to treat obesity, whether performed using open surgical techniques or laparoscopic or other less invasive techniques, will be generally referred to as “obesity surgery” or “obesity surgery procedures.”

Due to the stresses and changes to the digestive system caused by the various obesity surgery procedures, patients who undergo such procedures often suffer from one or more immediate post-operative and long term complications, such as malnutrition, dumping syndrome, dehydration, constipation, vomiting, nausea, and weight gain. Nausea and vomiting are the most common complications occurring in the first few months after obesity surgery. These symptoms may occur after eating too fast, drinking liquids while eating, not chewing enough, or eating more than the gastric pouch of significantly smaller post-surgical size can comfortably hold. Nausea and vomiting can also be triggered after trying new foods.

Dehydration is also an important concern in post-obesity surgery patients, especially if vomiting or diarrhea are frequent. Dehydration can be prevented by drinking water or low-calorie beverages between meals (when there is no food in the stomach). Since the size of the stomach is reduced after obesity surgery and can only hold approximately 3-4 ounces at a time, it is often quite challenging for post-operative patients to consume adequate amounts of food and liquid to maintain even minimally acceptable levels of nutrition and hydration.

Dumping syndrome is another common post-surgical complication and occurs when food passes too quickly from the stomach into the small intestine. Symptoms may include a combination of nausea, uncomfortable fullness, cramping, and diarrhea, or weakness, sweating, and fast heart rate. Dumping can be provoked, for example, by eating foods high in carbohydrate content (very sweet or sugary foods).

As just described, due to the many post-surgical symptoms faced by the typical obesity surgery patient, it is typically quite challenging for such patients to maintain adequate nutrition and hydration. Another challenge, over time, is that many patients continue to experience strong feelings of hunger and slowly increase the amount they eat as they slowly stretch out their stomachs. Such patients often regain much or all of the weight lost as a consequence of their gastric reduction procedures and are again faced with morbid obesity.

Yet another challenge for patients following obesity surgery procedures, is that they often fail to comply with their regimens of oral medication(s) required by one or more comorbid conditions. After most forms of obesity surgery, the size of the gastric pouch is radically reduced, so that post-operative patients may only be able to eat several bites of food or take several mouthfuls of liquid at a time. In many cases, even one large pill, let alone a handful of pills, may significantly fill a the gastric pouch of a post-op patient. Therefore, many post-obesity-surgery patients are faced with a choice, at mealtimes, of eating several bites of actual food or eating even less food and taking their prescribed oral medications. This difficult choice often leads to poor compliance with oral medication regimens, and the compliance problem is exacerbated by the fact that a patient may have to drink a few ounces of water with each pill. Poor compliance is often further compunded by post-operative complications such as vomiting, nausea, constipation, and all the other complications that make ingesting substances difficult. Even when post-op obesity patients do take their medications, such medications may not be adequately absorbed if the obesity surgery procedure included a malabsorptive component.

Many other medical and post-surgical conditions also involve difficult challenges of consuming sufficient and/or balanced nutrition while also complying with oral medication regimens. For example, diabetes patients typically have a restricted diet, and many diabetics also must consume insulin. Balancing their nutritional needs, appetites for certain foods, and insulin requirements is often challenging for many such patients, especially since the finger sticks and insulin injections required in diabetic regimens are painful and difficult to comply with fully. Other patients may have to take many medications every day, some of which, such as non-steroidal anti-inflammatories, should be taken with food to avoid damage to the stomach and/or to enhance absorption of the medication into the bloodstream. Elderly patients, demented patients, mentally challenged patients and others may often have difficulty remembering to take medications with food and may not remember or desire to take in adequate nutrition. Other patients who may have specific nutritional requirements and who may also be on one or more medications include patients with high blood pressure, high cholesterol, post-stroke, post-heart attack, anorexia, depression and the like.

Therefore, a need exists for a method and vehicle for supplying nutrients and pharmaceutical agents to patients who have undergone obesity surgery. Ideally, such a method and vehicle would help such patients consume adequate amounts of various important nutrients as well as one or more pharmaceutical agents, while also enhancing absorption of the nutrients and agent(s). Also ideally, the method and vehicle would help improve compliance with medical regimens and thus provide improved pharmacodynamics of oral medications and possibly lead to lower required doses of medications over time and/or shorter required courses of such medications. A need also exists for methods and vehicles for supplying nutrients and pharmaceutical agents to patients suffering from other medical or post-surgical conditions. At least some of these objectives will be met by the present invention.

### BRIEF SUMMARY OF THE INVENTION

Presented herein are food compositions including an effective amount of at least one pharmaceutical agent and a food carrier. In some embodiments, the pharmaceutical agent(s) in the food composition may exhibit an improved pharmacokinetic profile in patients who have undergone gastric bypass surgery, compared with the pharmacokinetic profile the same drug would exhibit if consumed alone, without being combined with the food carrier. In various embodiments, pharmaceutical agents incorporated in the food compositions may be directed at such conditions as hyperlipidemia, hypertension, diabetes, sleep apnea, degenerative joint disease, and/or depression. In some embodiments, the food carrier is designed to satisfy the specific nutritional requirements of a post-obesity-surgery patient. For example, the food carrier may have greater amounts of protein than of carbohydrate or fat, it may be designed to pass relatively slowly through the gastrointestinal tract to increase the time during which nutrients may be absorbed, it may include electrolytes, vitamins, minerals and the like, it may have an amount and blend of carbohydrate(s) to provide a relatively low osmolality and thus ameliorate dumping syndrome, and/or it may include an overall profile of carbohydrates, fats, proteins, vitamins, electrolytes and/or other nutrients to provide optimal nutrition to a post-surgical patient. By combining one or more pharmaceuticals with a specially designed food carrier, the food compositions of the present invention may achieve one or more of several objectives, such as: (1) to facilitate compliance of a post-obesity-surgery patient with an oral medication regimen; (2) to enhance absorption and/or efficacy of one or more pharmaceuticals; (3) to achieve and/or maintain an optimal nutritional intake and balance and thus possibly (4) reduce the need to take one or more medications over time, due to the enhanced nutrition of the patient; and (5) to help satisfy the psychological needs of the post-obesity-surgery patient by providing a food composition with a desirable taste, texture and smell that helps the patient feel full without overfilling the reduced gastric pouch. At least some of these objectives will be met by the food compositions of the present invention.

In one aspect of the present invention, a method of delivering nutrition and one or more pharmaceutical agents to a patient after obesity surgery to enhance at least one of the patient's nutritional intake or compliance with oral medication(s) may include: providing a food product in an amount suitable for fitting within a stomach of the patient after the obesity surgery, wherein the food product is formulated to include amounts of carbohydrate, fat, protein, vitamins, minerals and electrolytes to satisfy specific nutritional needs of the post-obesity-surgery patient; incorporating into the food product an effective amount of at least one pharmaceutical agent that modulates at least one of appetite, hyperlipidemia, hypertension, diabetes, sleep apnea, degenerative joint disease, or depression to produce a supplemented food product; and administering the supplemented food product to the patient after obesity surgery so as to enhance at least one of the patient's nutritional intake or compliance with oral medication.

In some embodiments, the food product may be provided in an amount between about 1 ounce and about 6 ounce. In some embodiments, the food product may be formulated to include a greater percentage of protein than of fat or carbohydrate. In some embodiments, the food product may be formulated to prolong its passage through a gastrointestinal tract of the patient, thus enhancing absorption of nutrition from the food product by the patient. In some embodiments, the food product may be formulated to include an amount and type of carbohydrate(s) to provide a relatively low osmolality and thus help alleviate dumping syndrome. In some embodiments, the food product may be formulated to slow gastric emptying time upon ingestion by the patient.

In some embodiments, the pharmaceutical agent may be absorbed into the patient's plasma at least 5% faster than the pharmaceutical agent would be absorbed if consumed alone, without being incorporated into the food product. In some embodiments, a maximum concentration of the pharmaceutical agent in the patient's plasma may be at least 1.5 times greater than the maximum concentration of the agent would be if consumed alone, without being incorporated into the food product. In some embodiments, a maximum concentration of the pharmaceutical agent in the patient's plasma may be obtained at least 1.5 times faster than it would be obtained if the agent were consumed alone, without being incorporated into the food product. In some embodiments, a half-life of the pharmaceutical agent in the patient's plasma may be at least 1.5 times longer than the half-life of the agent would be if consumed alone, without being incorporated into the food product.

In some embodiments, incorporating the pharmaceutical agent into the food product comprises homogeneously admixing the two. In some embodiments, the method may further include enteric coating the pharmaceutical agent before the incorporating step. In some embodiments, the supplemented food product, upon ingestion by the patient, may slow gastric emptying time.

In another aspect of the present invention, a method of providing a supplemented food product for enhancing a patient's nutritional intake and/or compliance with oral medication(s) after obesity surgery may involve: providing a food product in an amount suitable for fitting within a stomach of the patient after the obesity surgery, wherein the food product is formulated to include amounts of carbohydrate, fat, protein, vitamins, minerals and electrolytes to satisfy specific nutritional needs of the post-obesity-surgery patient; and incorporating into the food product an effective amount of at least one pharmaceutical agent that modulates at least one of appetite, hyperlipidemia, hypertension, diabetes, sleep apnea, degenerative joint disease, or depression to produce a supplemented food product.

Some embodiments may further include enteric coating the pharmaceutical agent before incorporating the agent into the food product. In some embodiments, the food product may be provided in an amount between about 1 ounce and about 6 ounces.

In another aspect of the present invention, a method of producing a pharmaceutically supplemented food product may include: providing a food product in an amount suitable for fitting within a stomach of the patient after the obesity surgery, wherein the food product is formulated to include amounts of carbohydrate, fat, protein, vitamins, minerals and electrolytes to satisfy specific nutritional needs of the post-obesity-surgery patient; and incorporating into the food product a daily dose of an effective amount of at least one pharmaceutical agent that modulates a condition associated with or caused by obesity.

In some embodiments, the condition associated with or caused by obesity may be selected from the group consisting of excessive appetite, hyperlipidemia, hypertension, diabetes, sleep apnea, degenerative joint disease and depression. In some embodiments, the effective amount of the pharmaceutical agent comprises an amount recommended by a physician, a pharmacologist, a nutritionist, or the Food and Drug Administration. A method of producing a pharmaceutically supplemented food product comprising incorporating into a food product a dose of at least one pharmaceutical agent that modulates a medical condition.

In another aspect of the present invention, a method for treating a patient may include providing a pharmaceutically supplemented food product containing a dose of at least one pharmaceutical agent that modulates a medical condition, wherein an effect of at least one of the food product and the pharmaceutical agent is enhanced by incorporating the food product and the pharmaceutical agent.

In another aspect of the present invention, a method for extending the life of a patent covering a pharmaceutical agent may include incorporating the pharmaceutical agent into a food product to enhance absorption of the pharmaceutical agent upon ingestion.

In another aspect of the present invention, a method for enhancing patient compliance with a prescription of a pharmaceutical agent to be consumed with food may involve: packaging the pharmaceutical agent in a package with an amount of food to consume with the agent; and administering the packaged pharmaceutical agent and food to the patient to enhance compliance.

In another aspect of the present invention, a food composition for enhancing compliance with oral medication(s) and/or nutritional intake in a patient after obesity surgery may include: an effective amount of at least one pharmaceutical agent to modulate at least one symptom of the patient after obesity surgery; and a food carrier in an amount suitable for fitting within a stomach of the patient after the obesity surgery and including amounts of carbohydrate, fat, protein, vitamins, minerals and electrolytes to satisfy specific nutritional needs of the post-obesity-surgery patient. The pharmaceutical agent may be homogeneously admixed in the food carrier, and the pharmaceutical agent in the food composition may exhibit an improved pharmacokinetic profile compared with the pharmaceutical agent administered alone.

In various embodiments, the food composition may comprise a liquid, semi-solid or solid. In some embodiments, the food composition may comprise a nutritional bar. In some embodiments, the effective amount of the at least one pharmaceutical agent may be less than or equal to a total daily dose of the at least one pharmaceutical agent approved by the Food and Drug Administration for treatment of the symptom via oral ingestion. In alternative embodiments, the effective amount of the at least one pharmaceutical agent may be less than or equal to a total daily dose of the at least one pharmaceutical agent recommended by a clinician for treatment of the symptom via oral ingestion.

In some embodiments, the pharmaceutical agent may be selected from the group consisting of statins, niacin, fibric acid derivatives, bile acid sequestrants, angiotensin converting enzyme inhibitors, angiotensin receptor blockers; beta-blockers, diuretics, calcium channel blockers, alpha-blockers, clonidine, minoxidil, sulfonylureas, meglitinides, biguanides, thiazolidinediones, alpha-glucosidase inhibitors, mirtazapine, acetaminophen, non-steroidal anti-inflammatory drugs, salicylates, muscle relaxants, chondritin and derivatives thereof, glucosamine and derivatives thereof, corticosteroids, hyaluronic acid, selective serotonin reuptake inhibitors, tricyclics, serotonin and norepinephrine reuptake inhibitors, norepinephrine and dopamine reuptake inhibitors, combined reuptake inhibitors and receptor blockers, and monamine oxidase inhibitors. In some embodiments, the at least one symptom may be selected from the group consisting of appetite, hyperlipidemia, hypertension, diabetes, sleep apnea, degenerative joint disease, and depression.

In another aspect of the present invention, a food composition for enhancing compliance with oral medication(s) and/or nutritional intake in a patient after obesity surgery may include: an effective amount of at least one pharmaceutical agent to modulate a condition associated with or caused by obesity; and a food carrier in an amount suitable for fitting within a stomach of the patient after the obesity surgery and including amounts of carbohydrate, fat, protein, vitamins, minerals and electrolytes to satisfy specific nutritional needs of the post-obesity-surgery patient. The pharmaceutical agent may be homogeneously admixed in the food carrier; and the food composition may comprise a prescribed daily dose of the pharmaceutical agent.

These and other aspects and embodiments of the present invention are described further below, in the Detailed Description of the Invention.

### INCORPORATION BY REFERENCE

All publications and patent applications mentioned in this specification are herein incorporated by reference to the same extent as if each individual publication or patent application were specifically and individually indicated to be incorporated by reference.

### DETAILED DESCRIPTION OF THE INVENTION

### Glossary of Terms

The phrase “pharmaceutical agent” refers to any agent which imparts a therapeutic effect and is used or indicated for use as a pharmaceutical. Pharmaceutical agents may be used in the treatment, diagnosis, modulation, or prevention of a disease state or symptom thereof. Exemplary pharmaceutical agents contemplated within the scope of the invention are provided in the following references (the disclosures of all of which are hereby incorporated by reference): Lippincott et al., Remington's Pharmaceutical Sciences: The Science and Practice of Pharmacy, 20th Ed., Williams and Wilkins Publishing, Baltimore (2000); and Lewis et al., Hawley's Condensed Chemical Dictionary, 14th Ed., John Wiley Publishing, New York (2001). Any additional pharmaceutical agents not listed in the foregoing references or not yet developed may also be incorporated into one or more embodiments of the present invention.

The phrase “effective amount” refers to any amount sufficient to achieve a desired activity or result. In relation to a pharmaceutical agent, effective amounts may, for example, be dosages that are recommended in the modulation of a disease state or symptom thereof. Effective amounts differ, depending on the pharmaceutical agent used and the route of administration employed. Effective amounts are routinely optimized, taking into consideration various factors of a particular patient, such as age, weight, gender, etc.

The term “modulates” refers to an ability to modify, attenuate, or alter a particular activity or state. In relation to a disease state or symptom thereof, modulation may, for example, be achieved by the use of an enzyme or protein antagonist, agonist, inhibitor, catalyst, or the like.

The phrase “pharmacokinetic profile” refers to various well known measurable factors used by one of ordinary skill in the art to determine the potency and efficacy of a particular pharmaceutical agent. Exemplary factors included in a pharmacokinetic profile of a particular agent are: plasma clearance (which can be measured in units of mL/kg/h); steady-state volume of distribution (which can be measured in units of L/kg); elimination, terminal, or plasma half-life (which can be measured in units of hrs); maximum plasma concentration (which can be measured in units of μg/mL); local or systemic bioavailability (which can be measured by percentage); mean accumulation ratio; inhibitory concentration for inhibition of a particular protein or enzyme, i.e., IC50 (which can be measured in units of μM or μg/mL); effective concentration for a particular activity of a particular protein or enzyme, i.e., EC50 (which can be measured in units of μM or μg/mL), and the like. Values for various pharmacokinetic profiles differ depending on the route of administration. See Goodman et al., The Pharmaceutical Basis of Therapeutics, 10th Ed., McGraw-Hill, New York (2001) for a more detailed discussion (the disclosure of which is hereby incorporated by reference).

As discussed previously, the terms “obesity surgery” and “obesity surgery procedures” generally refer to any restrictive, malabsorptive, restrictive/malabsorptive combination, or other surgical procedures directed at treating obesity.

The phrase “gastric bypass surgery” refers to a well known surgical procedure that decreases the size of the stomach and bypasses parts of the stomach and small intestines to reduce absorption of calories. The most common gastric bypass surgery is a Roux-en-Y gastric bypass where the stomach is made smaller by creating a small pouch at the top of the stomach using surgical staples or a plastic band. The smaller stomach is connected directly to the middle portion of the small intestine (jejunum), bypassing the rest of the stomach and the upper portion of the small intestine (duodenum). Gastric bypass surgery may be performed via an open incision in the abdomen laparoscopically.

The term “compliance” refers to a patient's willingness and/or ability to follow a prescribed course of medical treatment.

### Exemplary Advantages of Present Food Compositions

In various embodiments, the present food compositions are formulated for consumption by patients who have undergone obesity surgery, such as but not limited to gastric bypass surgery, and need pharmaceutical treatment for hyperlipidemia, hypertension, diabetes, sleep apnea, degenerative joint disease, and/or depression. Because patients who have undergone gastric bypass surgery have significantly reduced stomach sizes (the volume is typically reduced from about 1 quart to about 1 ounce, or 2 tablespoons), these patients are unable to consume normal amounts of food. As a consequence, a large number of patients who undergo gastric bypass surgery do not comply with intake of medications to treat other co-morbid conditions generally associated with obesity, such as hyperlipidemia, hypertension, diabetes, sleep apnea, degenerative joint disease, and depression. Noncompliance may be caused by a number of different factors, several of which were discussed previously, such as the small size of the patient's stomach and the desire to fill the small stomach with “real food” rather than oral medications. One advantage, therefore, of combining one or more pharmaceutical agents in a food carrier to provide the food compositions of the present invention may include enhanced compliance of post-obesity-surgery patients with regimens of oral medications/pharmaceutical agents.

In various embodiments, the present food compositions are formulated to include amounts of carbohydrates, fats, proteins, water soluble vitamins, electrolytes and/or the like to satisfy the specific nutritional requirements of the post-obesity-surgery patient. By “specific nutritional requirements,” it is meant one or more nutritional needs that a post-obesity-surgery patient may have due to the state of the patient after surgery. The food compositions may also be designed to have desirable taste, texture, viscosity, smell and/or the like to enhance a patient's pleasure in consuming such food compositions and thus satisfy the patient's psychological needs for consuming pleasing foodstuffs. For example, food carriers employed herein may mask the bitter taste associated with some pharmaceutical agents. In other embodiments, food carriers employed herein may mask undesirable textures or odors associated with some pharmaceutical agents. The present food compositions may be formulated to have any of a number of tastes, textures, smells and the like, which may be desirable for some patients.

In some embodiments, it may be possible to reduce a patient's reliance on or need for one or more pharmaceutical agents due to enhanced nutritional intake of the patient provided by the food composition. For example, in a diabetic patient, an optimal nutritional balance provided by a food composition may reduce and in some cases even eliminate the patient's need for insulin over a period of time. In some cases, the food composition may also reduce the patient's need over time by enhancing the patient's compliance with the pharmaceutical regimen and/or by enhancing absorption of the pharmaceuticals taken, such as by slowing gastric emptying and thus increasing the time the food composition (and thus the pharmaceutical agent) is in the patient's gastrointestinal system.

The size and volume of the present food compositions may be reduced in some embodiments to accommodate the smaller gastric pouch of patients who have undergone gastric bypass surgery. Thus, in some embodiments, food compositions are sized in volume to be about 6 ounces or less, and in some embodiments about 4 ounces or less, and in some embodiments about 1 ounce or less.

In a preferred embodiment, the present food compositions are designed, packaged, formulated, or indicated to be meal replacements for patients who have undergone gastric bypass surgery. Meal replacements according to the invention satisfy nutritional and pharmaceutical requirements. Such meal replacements may, for example, be sized about 6 ounces or less, and in some embodiments about 4 ounces or less, and in some embodiments about 1 ounce or less.

Changes in the gastric emptying time of patients who have undergone gastric bypass surgery are considered when formulating and optimizing the present food compositions. As a consequence of having a smaller stomach size, a patient who has undergone gastric bypass surgery may exhibit a reduced ability to absorb nutrients from the food they ingest. In one embodiment, when ingested, the present food compositions increase nutrient absorption from the food composition by slowing gastric emptying of the food.

In a preferred embodiment, pharmaceutical agents of the present food compositions exhibit an improved pharmacokinetic profile in patients who have undergone gastric bypass surgery in comparison to oral intake of the pharmaceutical agent alone. In one embodiment, the pharmaceutical agent of the present food compositions is absorbed into the patient's plasma at least about 5% faster than it would be absorbed if taken alone. In another embodiment, the maximum concentration of the pharmaceutical agent of the present food compositions in the patient's plasma is at least about 1.5 times greater than it would have been if the agent were taken alone. In yet another embodiment, the half-life of the pharmaceutical agent of the present food compositions in the patient's plasma is at least about 1.5 times longer than it would have been if the agent were taken alone.

### Representative Pharmaceutical Agents

A. Agents to Modulate Hyperlipidemia

Hyperlipidemia refers to a condition associated with elevated levels of lipids, such as cholesterol, cholesterol esters, phospholipids and triglycerides, in the bloodstream. Lipids are generally transported in the blood as part of lipoproteins. Lipoproteins are generally categorized into five major families: chylomicrons, very low-density lipoproteins (VLDL), intermediate-density lipoproteins (IDL), low-density lipoproteins (LDL), and high-density lipoproteins (HDL). Hyperlipidemia includes hypercholesterolemia, a condition associated with high cholesterol levels in the blood, and hypertriglyceridemia, a condition associated with high triglyceride levels in the blood. Representative pharmaceutical agents that are indicated for the treatment of hyperlipidemia include statins, bile acid resins, cholesterol absorption inhibitors, fibrates, probucol, and nicotinic acid.

Statins or HMG-CoA reductase inhibitors may be used according to the invention to modulate hyperlipidemia. Exemplary statin drugs currently available are atorvastatin (Lipitor™), cerivastatin (Baycol™), fluvastatin (Lescol™), lovastatin (Mevacor™), pravastatin (Pravachol™), and simvastatin (Zocor™). As a group, statins have been shown to decrease total and LDL cholesterol levels. Although all of the statins decrease triglyceride levels, only cerivastatin, atorvastatin and simvastatin are labeled by the U.S. Food and Drug Administration for this use. All statins exhibit a minimal effect in raising HDL levels; simvastatin and atorvastatin are labeled for this indication. In addition, all statins, except for cerivastatin, have been shown to be effective in reducing LDL levels.

Niacin may also be used to as an agent to modulate hyperlipidemia, and in particular, to lower blood lipid levels. Niacin has been shown to reduce serum triglyceride, total cholesterol, and LDL cholesterol values. Niacin also has the beneficial effect of raising HDL levels. An extended-release form of niacin (Niaspan™) has been developed which imparts the same beneficial lipid-altering effects as standard niacin. Although it is less effective than the statins in decreasing LDL levels, extended-release niacin can increase HDL values by 20 percent and decrease triglyceride levels by 25 percent.

Pharmaceutical agents that may be used to modulate hyperlipidemia also include fibric acid derivatives or fibrates, which can be used, in particular, to treat hypertriglyceridemia. Representative fibrates include clofibrate (Atromid-S™), gemfibrozil (Lopid™), and fenofibrate (Tricor™). Fibrates have been shown to decrease triglyceride values by 20 to 45 percent and increase HDL levels by 7 to 15 percent. Although fibrates generally lower LDL values by 10 to 20 percent, some patients with type IV hyperlipoproteinemia show an increased LDL level. Fibrates are contraindicated for patients with severe hepatic or renal dysfunction.

Bile acid sequestrants may also be used to modulate hyperlipidemia. Exemplary bile acid sequestrants currently available include cholestyramine (LoCholest™) and colestipol (Colestid™). These agents have been shown to lower LDL by approximately 20 percent and raise HDL by approximately 5 percent. On average, maximal therapeutic effect can be evident in patients one month after initiation of therapy.

B. Agents to Modulate Hypertension

Hypertension refers to a condition associated with high blood pressure, which is generally characterized as a condition where a patient's systolic blood pressure is consistently over 140 and/or diastolic blood pressure is consistently over 90. According to the invention, hypertension also embraces pre-hypertensive levels, such as when a patient's systolic blood pressure is between 120 and 139 or diastolic blood pressure is between 80 and 89 on multiple readings.

Hypertension itself may be caused by genetic and environmental factors or may be caused by another disorder (referred to sometimes as “secondary hypertension”). Secondary factors that can lead to elevated blood pressure include: adrenal gland tumors; Cushing's syndrome; kidney disorders, such as glomerulonephritis (inflammation of kidneys), renal vascular obstruction or narrowing, and renal failure; use of medications, drugs (such as oral contraceptives), or other chemicals; hemolytic-uremic syndrome; Henoch-Schonlein purpura; periarteritis nodosa; radiation enteritis; retroperitoneal fibrosis; and Wilm's tumor.

Pharmaceutical agents that may be used to modulate hypertension include angiotensin converting enzyme inhibitors (ACE inhibitors); angiotensin receptor blockers; beta-blockers; diuretics; calcium channel blockers (CCBs); alpha-blockers; clonidine, and minoxidil. Varying regimens and possible combinations of these agents may be employed depending on the age, gender, and health of the particular patient.

For example, for most elderly patients with hypertension, a small dosage of a diuretic (such as 12.5 mg of hydrochlorothiazide (Esidrix™)) is prescribed whereas for younger persons, a maximum of 25 mg per day of hydrochlorothiazide is generally prescribed. The combination of a thiazide and a potassium-sparing agent may also be employed.

For initial antihypertensive therapy, a beta blocker is more often prescribed than a diuretic in patients with angina. Patients with diabetes or diabetic nephropathy are often prescribed a diuretic in combination with an ACE inhibitor or angiotensin-II (A-II) receptor blocker. An alternative therapy for patients with isolated systolic hypertension who cannot take a diuretic or who respond poorly to diuretic therapy is a long-acting dihydropyridine calcium channel blocker. Also, combinations of a diuretic with a beta blocker, an ACE inhibitor, or an A-II receptor blocker may be more effective in some patients.

C. Agents to Modulate Diabetes

Diabetes refers to a condition associated with high blood glucose levels, and includes Type 1 and Type 2 diabetes. Pharmaceutical agents that may be used to modulate diabetes include insulin and other agents that lower blood glucose levels, such as sulfonylureas, meglitinides, biguanides, thiazolidinediones, and alpha-glucosidase inhibitors. Varying regimens and possible combinations of these agents may be employed depending on the age, gender, and health of the particular patient. These agents may be used in combination with each other to lower blood glucose levels by employing different biological mechanisms. For example, biguanide and sulfonylurea compounds are used together by some patients. Combining oral medications can enhance blood glucose control over taking one type of agent alone.

Sulfonylureas stimulate beta cells of the pancreas to release more insulin, and available sulfonylureas include chlorpropamide (Diabinese™), glipizide (Glucotrol™ and Glucotrol-XL™), glyburide (Micronase™, Glynase™, and Diabeta™), and glimepiride (Amaryl™). All sulfonylurea drugs generally have similar effects on blood glucose levels, but differ in side effects, how often they are taken, and interactions with other drugs.

Meglitinides also stimulate beta cells to release insulin but have chemical structures unique from sulfonylureas. Exemplary meglitinides include repaglinide (Prandin™) and nateglinide (Starlix™).

Biguanides lower blood glucose levels primarily by decreasing the amount of glucose produced by the liver. Metformin (Glucophage™) is an exemplary type of biguanide. Metformin also helps to lower blood glucose levels by making muscle tissue more sensitive to insulin so glucose can be absorbed.

Thiazolidinediones may also be used to modulate diabetes by helping insulin impart more lasting effects in muscle and fat tissue, and by reducing glucose production in the liver. Exemplary thiazolidinediones include rosiglitazone (Avandia™), troglitazone (Rezulin™), and pioglitazone (ACTOS™). Thiazolidinediones are effective in lowering blood glucose levels, but can also adversely affect the liver.

Alpha-glucosidase inhibitors may also be used to modulate diabetes by blocking the breakdown of starches, such as bread, potatoes, and pasta in the intestine. Alpha-glucosidase inhibitors also slow the breakdown of some sugars, such as table sugar, and thereby decrease the rise in blood glucose levels after consumption of a meal. Exemplary Alpha-glucosidase inhibitors currently available include acarbose (Precose™) and meglitol (Glyset™).

D. Agents to Modulate Sleep Apnea

Sleep apnea is condition associated with cessation of breathing during sleep that is generally caused by repetitive partial or complete obstruction of the airway by pharyngeal structures. Patients with obstructive sleep apnea often are overweight, snore loudly, and complain of daytime fatigue and sleepiness.

Generally, patients with sleep apnea are prescribed devices that employ continuous positive airway pressure or undergo surgery to improve obstructed breathing pathways. However, recent studies have shown that certain antidepressants, such as mirtazapine, are viable as oral agents to modulate sleep apnea. Mirtazapine attenuates brain serotonin levels, and modulation of serotonin seems to help patients breathe normally during sleep. Mirtazapine is approved by the FDA for treating depression, and has not yet been indicated for treatment of sleep apnea.

E. Agents to Modulate Degenerative Joint Disease

Degenerative joint disease is a condition associated with degeneration of cartilage and overgrowth of bone in joints and is typically accompanied by minimal inflammation. Degenerative joint disease includes osteoarthritis and osteoarthrosis.

Exemplary compounds that are typically used to modulate the symptoms of degenerative joint disease include acetaminophen (Tylenol™), non-steroidal anti-inflammatory drugs (NSAID) such as ibuprofen, salicylates, muscle relaxants, chondritin and derivatives thereof, glucosamine and derivatives thereof, corticosteroids, and hyaluronic acid.

F. Agents to Modulate Depression

There are numerous pharmaceutical agents well known to one of ordinary skill in the art that can be used to modulate depression. Exemplary agents that may be used according to the invention include, but are not limited to, selective serotonin reuptake inhibitors, tricyclics, serotonin and norepinephrine reuptake inhibitors, norepinephrine and dopamine reuptake inhibitors, combined reuptake inhibitors and receptor blockers, and monamine oxidase inhibitors.

Exemplary antidepressant that can be used according to the invention include fluoxetine (N-methyl-3-(p-trifluoromethylphenoxy)-3-phenylpropylamine), duloxetine (N-methyl-3-(1-naphthalenyloxy)-3-(2-thienyl)propanamine), venlafaxine, milnacipran (N,N-diethyl-2-aminomethyl-1-phenylcyclopropanecarboxamide), citalopram (1-[3-(dimethylamino)propyl]-1-(4-fluorophenyl)-1,3-dihydro-5-isobenzofurancarbonitrile), fluvoxamine (5-methoxy-1-[4-(trifluoromethyl)phenyl]-1-pentanone O-(2-aminoethyl)oxime), paroxetine (trans-(−)-3-[(1,3-benzodioxol-5-yloxy)methyl]-4-(4-fluorophenyl)piperidine), sertraline ((1S-cis)-4-(3,4-dichlorophenyl)-1,2,3,4-tetrahydro-N-methyl-1-naphthylamine).

Additional representative antidepressants include citalopram (Celexa™), escitalopram (brand name: Lexapro™), fluoxetine (Prozac™), paroxetine (Paxil™, Pexeva™), sertraline (Zoloft™), amitriptyline (Elavil™), desipramine (Norpramin™), imipramine (Tofranil™), nortriptyline (Aventyl™, Pamelor™), venlafaxine (Effexor™), duloxetine (Cymbalta™), bupropion (Wellbutrin™), trazodone (Desyrel™), nefazodone (Serzone™), maprotiline, mirtazpine (Remeron™), isocarboxazid (Marplan™), phenelzine (Nardil™), and tranlcypromine (Parnate™).

Any of the above-listed pharmaceutical agents may be enteric coated before incorporation of the agent(s) with a food carrier to provide a food composition, according to various embodiments of the invention. Enteric coating is well known by those skilled in the art, and any suitable method for enteric coating may be used, in various embodiments.

### Exemplary Food Carriers

In various embodiments, food carriers are designed to have various nutritional profiles to meet the specific needs of one or more types of patients. For example, in some embodiments, food carriers may be designed to include a profile of carbohydrates, fats and proteins in a small amount of food (for example from about 1 ounce to about 6 ounces) to meet the specific nutritional needs of patients immediately after obesity surgery. In alternative embodiments, food carriers may have alternative combinations of carbohydrates, fats and proteins to meet the nutritional needs of patients several weeks, months or even years after obesity surgery. In yet other alternative embodiments, food carriers may be specifically designed to meet the nutritional needs of other types of patients, such as elderly patients, diabetic patients, anorexic patients, cachectic patients, cancer patients or the like. In some embodiments, the nutritional characteristics of the food carriers may not only help meet specific nutritional needs of a patient, but may also enhance the absorption of nutrients, slow gastric emptying to prolong feelings of fullness, enhance the absorption of one or more pharmaceutical agents combined with the food carrier, and/or the like.

In some embodiments, food carriers are formulated to have suitable and desirable taste, texture, and viscosity for consumption, in addition to the nutritional characteristics discussed above. Any suitable food carrier may be used in various embodiments of the present food compositions. In one embodiment, food carriers employed herein may mask the bitter taste associated with some pharmaceutical agents. By selecting particular food carriers, food compositions presented herein exhibit more desirable textures and aromas than that of pharmaceutical agents. Additionally, the present food compositions may be formulated to have a desired viscosity to facilitate digestion in patients who have undergone gastric bypass surgery.

In one embodiment, for example, liquid food carriers may be used to obtain the present food compositions in the form of beverages, such as supplemented juices, coffees, teas, and the like. In other embodiments, solid food carriers may be used according to the invention to obtain the present food compositions in the form of meal replacements, such as supplemented snack bars, pasta, breads, and the like. In yet other embodiments, semi-solid food carriers may be used according to the invention to obtain the present food compositions in the form of gums, chewy candies or snacks, melt-in-the-mouth strips or lozenges and the like.

In some embodiments, one or more pharmaceutical agents may be enteric coated before combining with a food carrier to provide a food composition. Such pharmaceutical agents may be broken down into small pieces or fragments before enteric coating, and such enteric coated pieces may then be embedded, mixed or otherwise combined with the food carrier to provide the food composition.

In one example, a regimen may be provided for a post-operative obesity surgery patient. The regimen may include one or more pharmaceutical agents incorporated into a liquid food carrier, which combined liquid/pharmaceutical combination may be consumed for a prescribed number of weeks post-surgery, as the patient initially recovers. The regimen may next include a semi-solid or gel food carrier, such as a dissolving lozenge or strip that melts on the tongue, combined with one or more pharmaceutical agents. This semisolid/pharmaceutical combination may be consumed for a prescribed number of weeks after the liquid regimen is completed. The regimen may next include a solid food carrier, such as an energy bar, combined with one or more pharmaceutical agents, and this combined solid food/pharmaceutical may be consumed for a prescribed number of weeks after the semi-solid regimen is completed. In some embodiments, multiple different solid food/pharmaceutical combinations may be provided. In some embodiments, different solid food/pharmaceutical combinations may be provided as a sequence.

In another example, a food carrier may comprise a concentrated energy bar-like substance having a nutritional profile of carbohydrates, proteins and fats specifically designed for post-obesity surgery patients. For example, in some embodiments, such a bar food carrier may be from about 1 ounce to about 6 ounces, to fit in a small post-surgical gastric pouch. In some embodiments, the bar, upon ingestion by the patient, may slow gastric emptying time. Slowed gastric emptying time may be achieved, for example, by a bar having a high protein and/or fat content and relatively little carbohydrate. Such a bar may occupy space in the gastric pouch, thus prolonging feelings of satiety, and/or may partially block passage of food through the gastroduodenal sphincter, which may help enhance weight loss.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various embodiments may be included in some embodiments and not in others. These and many other modifications may be made to many of the described embodiments. Therefore, the foregoing description is provided primarily for exemplary purposes and should not be interpreted to limit the scope of the invention as it is set forth in the claims.

What is claimed is:

1. A method of delivering nutrition and one or more pharmaceutical agents to a patient after obesity surgery to enhance at least one of the patient's nutritional intake or compliance with oral medication(s), the method comprising:
(a) providing a food product in an amount suitable for fitting within a stomach of the patient after the obesity surgery, wherein the food product is formulated to include amounts of carbohydrate, fat, protein, vitamins, minerals and electrolytes to satisfy specific nutritional needs of the post-obesity-surgery patient;
(b) incorporating into the food product an effective amount of at least one pharmaceutical agent that modulates at least one of appetite, hyperlipidemia, hypertension, diabetes, sleep apnea, degenerative joint disease, or depression to produce a supplemented food product; and
(c) administering the supplemented food product to the patient after obesity surgery so as to enhance at least one of the patient's nutritional intake or compliance with oral medication.

2. The method of claim 1, wherein the food product is provided in an amount between about 1 ounce and about 6 ounces.

3. The method of claim 1, wherein the food product is formulated to include a greater percentage of protein than of fat or carbohydrate.

4. The method of claim 1, wherein the food product is formulated to prolong its passage through a gastrointestinal tract of the patient, thus enhancing absorption of nutrients from the food product by the patient.

5. The method of claim 1, wherein the food product is formulated to include an amount and type of carbohydrate(s) to provide a relatively low osmolality and thus help alleviate dumping syndrome.

6. The method of claim 1, wherein the food product is formulated to slow gastric emptying time upon ingestion by the patient.

7. The method of claim 1, wherein the supplemented food product is formulated such that the pharmaceutical agent is absorbed into the patient's plasma at least 5% faster than the pharmaceutical agent would be absorbed if consumed alone, without being incorporated into the food product.

8. The method of claim 1, wherein the supplemented food product is formulated such that a maximum concentration of the pharmaceutical agent in the patient's plasma is at least 1.5 times greater than the maximum concentration of the agent would be if consumed alone, without being incorporated into the food product.

9. The method of claim 1, wherein the supplemented food product is formulated such that a maximum concentration of the pharmaceutical agent in the patient's plasma is obtained at least 1.5 times faster than it would be obtained if the agent were consumed alone, without being incorporated into the food product.

10. The method of claim 1, wherein the supplemented food product is formulated such that a half-life of the pharmaceutical agent in the patient's plasma is at least 1.5 times longer than the half-life of the agent would be if consumed alone, without being incorporated into the food product.

11. The method of claim 1, wherein incorporating comprises homogeneously admixing.

12. The method of claim 1, further comprising applying an enteric coating to the pharmaceutical agent before the incorporating step.

13. A method of providing a supplemented food product for enhancing at least one of a patient's nutritional intake or compliance with oral medication(s) after obesity surgery, the method comprising:
(a) providing a food product in an amount suitable for fitting within a stomach of the patient after the obesity surgery, wherein the food product is formulated to include amounts of carbohydrate, fat, protein, vitamins, minerals and electrolytes to satisfy specific nutritional needs of the post-obesity-surgery patient; and
(b) incorporating into the food product an effective amount of at least one pharmaceutical agent that modulates at least one of appetite, hyperlipidemia, hypertension, diabetes, sleep apnea, degenerative joint disease, or depression to produce a supplemented food product.

14. The method of claim 13, wherein the food product is provided in an amount between about 1 ounce and about 6 ounces.

15. The method of claim 13, wherein the food product is formulated to include a greater percentage of protein than of fat or carbohydrate.

16. The method of claim 13, wherein the food product is formulated to prolong its passage through a gastrointestinal tract of the patient, thus enhancing absorption of nutrition from the food product by the patient.

17. The method of claim 13, wherein the food product is formulated to include an amount and type of carbohydrate(s) to provide a relatively low osmolality and thus help alleviate dumping syndrome.

18. The method of claim 13, wherein the food product is formulated to slow gastric emptying time upon ingestion by the patient.

19. A method of producing a pharmaceutically supplemented food product, the method comprising:
(a) providing a food product in an amount suitable for fitting within a stomach of the patient after the obesity surgery, wherein the food product is formulated to include amounts of carbohydrate, fat, protein, vitamins, minerals and electrolytes to satisfy specific nutritional needs of the post-obesity-surgery patient; and
(b) incorporating into the food product a daily dose of an effective amount of at least one pharmaceutical agent that modulates a condition associated with or caused by obesity to provide a supplemental food product formulated to enhance at least one of the patient's nutritional intake or compliance with oral medication.

20. The method of claim 19, wherein the condition associated with or caused by obesity is selected from the group consisting of excessive appetite, hyperlipidemia, hypertension, diabetes, sleep apnea, degenerative joint disease and depression.

21. The method of claim 19, wherein the effective amount of the pharmaceutical agent comprises an amount recommended by a physician, a pharmacologist, a nutritionist, or the Food and Drug Administration.

22. The method of claim 19, wherein incorporating comprises homogeneously admixing the pharmaceutical agent into the food carrier.

23. A method of producing a pharmaceutically supplemented food product comprising incorporating into a food product a dose of at least one pharmaceutical agent that modulates a medical condition.

24. A method for enhancing nutritional uptake and/or drug compliance in a patient comprising providing a pharmaceutically supplemented food product containing a dose of at least one pharmaceutical agent that modulates a medical condition, wherein an effect of at least one of the food product and the pharmaceutical agent is enhanced by incorporating the food product and the pharmaceutical agent.

25. A method for securing intellectual property rights covering a pharmaceutical agent, the method comprising incorporating the pharmaceutical agent into a food product to enhance treatment of a medical condition, wherein the food product with incorporated pharmaceutical agent treats the medical condition more effectively than the food product alone or the pharmaceutical agent alone.

26. A method for enhancing patient compliance with a prescription of a pharmaceutical agent to be consumed with food, the method comprising:
(a) packaging the pharmaceutical agent in a package with an amount of food to consume with the agent; and
(b) administering the packaged pharmaceutical agent and food to the patient to enhance compliance.

27. A food composition for enhancing compliance with oral medication(s) and/or nutritional intake in a patient after obesity surgery, the food composition comprising:
(a) an effective amount of at least one pharmaceutical agent to modulate at least one symptom of the patient after obesity surgery; and
(b) a food carrier in an amount suitable for fitting within a stomach of the patient after the obesity surgery and including amounts of carbohydrate, fat, protein, vitamins, minerals and electrolytes to satisfy specific nutritional needs of the post-obesity-surgery patient;
wherein the pharmaceutical agent is homogeneously admixed in the food carrier, and
wherein the pharmaceutical agent in the food composition exhibits an improved pharmacokinetic profile compared with the pharmaceutical agent administered alone.

28. The food composition of claim 27, wherein the food composition comprises a liquid.

29. The food composition of claim 27, wherein the food composition comprises a semi-solid.

30. The food composition of claim 27, wherein the food composition comprises a solid.

31. The food composition of claim 27, wherein the food composition comprises a nutritional bar.

32. The food composition of claim 27, wherein the effective amount of the at least one pharmaceutical agent is less than or equal to a total daily dose of the at least one pharmaceutical agent approved by the Food and Drug Administration for treatment of the symptom via oral ingestion.

33. The food composition of claim 27, wherein the effective amount of the at least one pharmaceutical agent is less than or equal to a total daily dose of the at least one pharmaceutical agent recommended by a clinician for treatment of the symptom via oral ingestion.

34. The food composition of claim 27, wherein the pharmaceutical agent is selected from the group consisting of statins, niacin, fibric acid derivatives, bile acid sequestrants, angiotensin converting enzyme inhibitors, angiotensin receptor blockers; beta-blockers, diuretics, calcium channel blockers, alpha-blockers, clonidine, minoxidil, sulfonylureas, meglitinides, biguanides, thiazolidinediones, alpha-glucosidase inhibitors, mirtazapine, acetaminophen, non-steroidal anti-inflammatory drugs, salicylates, muscle relaxants, chondritin and derivatives thereof, glucosamine and derivatives thereof, corticosteroids, hyaluronic acid, selective serotonin reuptake inhibitors, tricyclics, serotonin and norepinephrine reuptake inhibitors, norepinephrine and dopamine reuptake inhibitors, combined reuptake inhibitors and receptor blockers, and monamine oxidase inhibitors.

35. The food composition of claim 27, wherein the at least one symptom is selected from the group consisting of appetite, hyperlipidemia, hypertension, diabetes, sleep apnea, degenerative joint disease and depression.

36. A food composition for enhancing compliance with oral medication(s) and/or nutritional intake in a patient after obesity surgery, the food composition comprising:
(a) an effective amount of at least one pharmaceutical agent to modulate a condition associated with or caused by obesity; and
(b) a food carrier in an amount suitable for fitting within a stomach of the patient after the obesity surgery and including amounts of carbohydrate, fat, protein, vitamins, minerals and electrolytes to satisfy specific nutritional needs of the post-obesity-surgery patient;
wherein the pharmaceutical agent is homogeneously admixed in the food carrier; and
wherein the food composition comprises a prescribed daily dose of the pharmaceutical agent.

37. The food composition of claim 36, wherein the food composition comprises a liquid.

38. The food composition of claim 36, wherein the food composition comprises a semi-solid.

39. The food composition of claim 36, wherein the food composition comprises a solid.

40. The food composition of claim 39, wherein the food composition comprises a nutritional bar.

41. The food composition of claim 36, wherein the effective amount of the at least one pharmaceutical agent is less than or equal to a total daily dose of the at least one pharmaceutical agent approved by the Food and Drug Administration for treatment of the symptom via oral ingestion.

42. The food composition of claim 36, wherein the effective amount of the at least one pharmaceutical agent is less than or equal to a total daily dose of the at least one pharmaceutical agent recommended by a clinician for treatment of the symptom via oral ingestion.

43. The food composition of claim 36, wherein the pharmaceutical agent is selected from the group consisting of statins, niacin, fibric acid derivatives, bile acid sequestrants, angiotensin converting enzyme inhibitors, angiotensin receptor blockers; beta-blockers, diuretics, calcium channel blockers, alpha-blockers, clonidine, minoxidil, sulfonylureas, meglitinides, biguanides, thiazolidinediones, alpha-glucosidase inhibitors, mirtazapine, acetaminophen, non-steroidal anti-inflammatory drugs, salicylates, muscle relaxants, chondritin and derivatives thereof, glucosamine and derivatives thereof, corticosteroids, hyaluronic acid, selective serotonin reuptake inhibitors, tricyclics, serotonin and norepinephrine reuptake inhibitors, norepinephrine and dopamine reuptake inhibitors, combined reuptake inhibitors and receptor blockers, and monamine oxidase inhibitors.

44. The food composition of claim 36, wherein the condition is selected from the group consisting of excessive appetite, hyperlipidemia, hypertension, diabetes, sleep apnea, degenerative joint disease, and depression.
